(19) United States
(12) Patent Application Publication
(10) Pub. No.: US 2009/0286959 A1
(43) Pub. Date: Nov. 19, 2009

(54) High Molecular Weight Fibroin Having Improved Antioxidant Activity, Tyrosinase Inhibitory Ability and/or Cytotoxicity to Cancer Cells by Irradiation, and Methods of Making and Using the Same

(75) Inventors: Ju-Woon LEE, Jeollauk-Do (KR); Jong-Il Choi, Seoul (KR); Jae-Hun Kim, Jeollabuk-Do (KR); Beom-Seok Song, Seoul (KR); Myung-Woo Byun, Jeollabuk-Do (KR); Eui-Baek Byun, Jeollabuk-Do (KR); Nak-Yun Sung, Chungcheongnam-do (KR)

Correspondence Address: STERNE, KESSLER, GOLDSTEIN & FOX P.L.L.C., 1100 NEW YORK AVENUE, N.W., WASHINGTON, DC, 20005, US

(73) Assignee: Korea Atomic Energy Research Institute, Daejeon (KR)

(21) Appl. No.: 12/468,215

(22) Filed: May 19, 2009

(30) Foreign Application Priority Data
May 19, 2008 (KR) 10-2008-0046311

(51) Int. Cl. C07K 14/435

(52) U.S. Cl. 530/353

(57) ABSTRACT

Disclosed are a fibroin with increased molecular weight and improved antioxidant activity, tyrosinase inhibitory ability and cytotoxicity to cancer cells, which has a molecular structure modified by irradiation, a method for production thereof, use of the irradiated fibroin in various applications for enhancing antioxidant activity, tyrosinase inhibitory ability and/or cytotoxicity to cancer cells such as foods, cosmetics, medicines, etc.

### CROSS-REFERENCE TO RELATED APPLICATIONS

This application claims priority to Korean Patent Application No. 10-2008-0046311, filed on May 19, 2008, in the Korean Intellectual Property Office, the entire contents of which are hereby incorporated by reference.

### BACKGROUND OF THE INVENTION

1. Field of the Invention

The present invention relates to high molecular weight fibroin having improved antioxidant activity, tyrosinase inhibitory ability and/or cytotoxicity to cancer cells by irradiation, its production method and use thereof, more particularly, to a fibroin with increased molecular weight and improved antioxidant activity, tyrosinase inhibitory ability and cytotoxicity to cancer cells, which has a molecular structure modified by irradiation, a method for production thereof, use of the irradiated fibroin in various applications for enhancing antioxidant activity, tyrosinase inhibitory effects and/or cytotoxicity to cancer cells such as foods, cosmetics, medicines, etc. and a composition containing the irradiated fibroin as an active ingredient.

2. Description of the Related Art

Silk protein has been used for manufacturing clothing for many years. However, only recently has research and investigation into biochemical and medical applications of such a silk protein been conducted. As a result, silk protein has drawn considerable attention as a novel biomaterial. A variety of physiological activities of the silk fibroin have been reported in the art. For example, it was disclosed that a rat provided with silk fibroin shows lowered blood cholesterol levels and blood glucose levels while alcohol absorption was inhibited (see J. Luo et al., “Study on foodization of fibroin and its functionality,” 2nd International Silk Conference, Beijing, China, p. 73 (1993)). In another case, a fibroin reacted using sulfate groups exhibited anti-HIV activity (K. Gotoh et al., “Sulfated fibroin, a novel sulfated peptide derived from silk, inhibits human immunodeficiency virus replication in vitro,” Biosci. Biotechnol. Biochem. 64:1664 (2000)).

However, in spite of such enormous studies and eco-friendly silk proteins, investigations into improvement in performance of the fibroin used for beneficial applications such as functional foods, cosmetics, medicines, and so forth have yet to be proposed in the art.

Accordingly, there is still a strong need for development of techniques for enhancing functions and/or performance of fibroin as described above, which is capable of being used in practical applications such as foods or other products.

### BRIEF SUMMARY OF THE INVENTION

The present inventors have conducted extensive research in regard to fibroin with improved physiological activity and found that an irradiated fibroin solution has a modified molecular structure and a high molecular weight due to irradiation, and exhibits improved radial scavenging ability, tyrosinase inhibitory ability and/or cytotoxicity to cancer cells, thereby completing the present invention.

Accordingly, an object of the present invention is to provide a fibroin with improved physiological activity, which has a molecular structure modified by irradiation.

Another object of the present invention is to provide a method for production of a fibroin with improved physiological activity, which has a molecular structure modified by irradiation.

Another object of the present invention is to provide a method for using a fibroin with improved physiological activity, which has a molecular structure modified by irradiation.

A further object of the present invention is to provide a composition including a fibroin with improved physiological activity, which has a molecular structure modified by irradiation, as an active ingredient.

In order to accomplish the above objects of the present invention, there is provided a fibroin having a molecular structure modified by irradiation, which has increased molecular weight and improved antioxidant activity, tyrosinase inhibitory ability and/or cytotoxicity to cancer cells.

The present invention also provides a method for production of a fibroin having a molecular structure modified by irradiation, which has increased molecular weight and improved antioxidant activity, tyrosinase inhibitory ability and/or cytotoxicity to cancer cells, including: irradiating an initial fibroin so as to reach a radiation absorbed dose ranging from 1 kiloGray (“kGy”) to 1,000 kGy.

The present invention also provides a method for using the fibroin with modified molecular structure as described above, as a raw material in any one of foods, cosmetics and medicines for enhancing antioxidant activity, tyrosinase inhibitory ability and/or cytotoxicity to cancer cells.

The present invention further includes a composition including the fibroin with modified molecular structure as an active ingredient, which is useful for enhancing antioxidant activity, tyrosinase inhibitory ability and/or cytotoxicity to cancer cells. In some embodiments, the composition comprises a modified fibroin of the present invention, wherein the modified fibroin has a molecular weight of 10 kDa to 1,000 kDa and has a molecular structure that includes less than 50% of an α-helix structure, as determined by circular dichroism. In some embodiments, the composition comprises a modified fibroin having a molecular weight of 200 kDa to 1,000 kDa and has a molecular structure that includes 30% or more of a β-sheet structure and 30% or more of a random coil structure, as determined by circular dichroism.

A high molecular weight fibroin with a molecular structure modified by γ-irradiation according to the present invention has superior physiological characteristics such as improved radical scavenging effects, tyrosinase inhibitory effects and/or cytotoxicity to cancer cells, as well as favorable whitening effects, compared to conventional materials without irradiation, thereby being effectively used as raw materials for foods, cosmetics, medicines, and the like.

### BRIEF DESCRIPTION OF THE DRAWINGS

These and other objects, features, aspects, and advantages of the present invention will be more fully described in the following detailed description of preferred embodiments, taken in conjunction with the accompanying drawings. The accompanying drawings, which are incorporated herein and form a part of the specification, illustrate one or more embodiments of the present invention and, together with the description, further serve to explain the principles of the invention and to enable a person skilled in the pertinent art to make and use the invention. In the drawings:

FIG. 1 depicts graphs illustrating UV absorption spectrum results of a fibroin protein obtained by γ-irradiation;

FIG. 2 depicts graphs illustrating far-UV CD spectrum results of a fibroin protein obtained by γ-irradiation in order to analyze secondary structure of the fibroin protein;

FIG. 3 depicts graphs illustrating GPC measured results of molecular weight of a fibroin protein obtained by γ-irradiation;

FIG. 4 depicts graphs illustrating improved radical scavenging results of a fibroin protein obtained by γ-irradiation;

FIG. 5 depicts graphs illustrating improved tyrosinase inhibitory effect results of a fibroin protein obtained by γ-irradiation;

FIGS. 6A-6C illustrate increases in improved cytotoxicity results of a fibroin protein obtained by γ-irradiation to cancer cells; especially, FIG. 6A shows improved cytotoxicity results in HT-29 cell-line as colon cancer cells, FIG. 6B shows improved cytotoxicity results in B16BL6 cell-line as melanoma cells, and FIG. 6C shows improved cytotoxicity results in AGS cell-line as gastric cancer cells; and

FIG. 7 depicts graphs illustrating reduced cytotoxicity results of a fibroin protein obtained by γ-irradiation to normal cells.

One or more embodiments of the present invention will now be described with reference to the accompanying drawings. In the drawings, like reference numbers can indicate identical or functionally similar elements.

### DETAILED DESCRIPTION OF THE INVENTION

This specification discloses one or more embodiments that incorporate the features of this invention. The disclosed embodiment(s) merely exemplify the invention. The scope of the invention is not limited to the disclosed embodiment(s). The invention is defined by the claims appended hereto.

The embodiment(s) described, and references in the specification to “one embodiment,” “an embodiment,” “an example embodiment,” etc., indicate that the embodiment(s) described can include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it is understood that it is within the knowledge of one skilled in the art to effect such feature, structure, or characteristic in connection with other embodiments whether or not explicitly described.

Hereinafter, the present invention will be described in greater detail.

In an aspect of the present invention to accomplish the above objects, there is a fibroin having a molecular structure modified by irradiation, which has increased molecular weight and exhibits improved antioxidant activity, tyrosinase inhibitory ability and/or cytotoxicity to cancer cells.

The fibroin used herein can be obtained from cocoons. More particularly, treating a cocoon with a sodium carbonate solution can remove sericin to prepare a fibroin solution, followed by removing impurities therefrom through dialysis. The fibroin solution obtained after removing the impurities is preferably subjected to lyophilization to produce a final fibroin in the form of powder.

As used herein, “irradiation” refers to exposure to radiation, and the terms can be used interchangeably. Irradiation can include at least one form of radiation selected from: γ-rays, an electron beam, and x-rays. In some embodiments, the radiation is γ-irradiation or electron beam irradiation due to the substantial increase in molecular weight of fibroin caused by these forms of irradiation.

A radiation absorbed dose can range from 1 kGy to 1,000 kGy, 5 kGy to 500 kGy, 5 kGy to 200 kGy, 5 kGy to 150 kGy, 5 kGy to 100 kGy, 100 kGy to 1,000 kGy, 100 kGy to 500 kGy, or 100 kGy to 200 kGy. Generally, absorbed doses of radiation that are less than 1 kGy are insufficient to provide the desired changes in the fibroin. Conversely, when the radiation absorbed dose exceeds 1,000 kGy, a problem of material decomposition can be caused by such a high dose of radiation.

The modified fibroin of the present invention (fibroin treated by irradiation according to the present invention) shows an increase in optical density of 105% or more at 280 nm, and 500% or more at 300 nm in the UV absorption spectrum, compared to a typical fibroin without irradiation.

Modification of the molecular structure of the fibroin according to the present invention can be a decrease in α-helix secondary structure, or otherwise, an increase in at least one secondary structure selected from: β-sheet, β-turn, and random coil structures. Modification of the molecular structure can be determined by analytical techniques known to a person of ordinary skill in the art, for example, circular dichroism.

In some embodiments, the modified fibroin of the present invention exhibits a relative decrease in α-helix structure, as determined by circular dichroism, of about 40% or more, 50% or more, 60% or more, 75% or more, or 80% or more, compared to fibroin that has not been irradiated.

In some embodiments, the modified fibroin of the present invention exhibits a relative increase in β-sheet structure, as determined by circular dichroism, of 50% or more, 60% or more, 75% or more, or 100% or more, compared to fibroin that has not been irradiated.

In some embodiments, the modified fibroin of the present invention exhibits a relative increase in random coil structure, as determined by circular dichroism, of 50% or more, 60% or more, 75% or more, 100% or more, or 125% or more, compared to fibroin that has not been irradiated.

The modification of the molecular structure of the fibroin according to the present invention can be a decrease in α-helix secondary structure as well as an increase in at least one secondary structure selected from a group consisting of β-sheet, β-turn and random coil structures.

More preferably, the modification of the molecular structure of the fibroin according to the present invention is a decrease in α-helix secondary structure and, at the same time, an increase in each of β-sheet, β-turn and random coil type secondary structures, in view of maximum improvement in antioxidant activity, tyrosinase inhibitory ability and cytotoxicity to cancer cells.

Lee et al. (“Effect of gamma-irradiation on the physicochemical properties of porcine and bovine blood plasma proteins,” Food Chem. 82:521 (2003)) has reported that irradiation of a protein in a solution phase can generate oxygen radicals to easily break covalent bonds of the protein which in turn can collapse an aligned structure thereof, resulting in modification of secondary and tertiary structures in the protein.

The β-turn structure which includes four (4) residual groups to form a hydrogen bond between i-th carbonyl group and i+3th amine group in the protein, is a general element constituting a globular protein and can be often detected on a surface of the globular protein. Such β-turn structure can reverse the direction of a polypeptide chain so as to promote protein folding. Therefore, the β-turn can play an important role in natural protein folding.

The irradiation-modified fibroin of the present invention can have a molecular weight ranging from 5 kDa to 2,000 kDa, 5 kDa to 1,000 kDa, or 10 kDa to 1,000 kDa. As described above, in consideration of a typical fibroin without irradiation, which has a molecular weight of not more than 5 kDa, it is identified that the molecular weight of the present inventive fibroin is considerably increased. In some embodiments, the modified fibroin of the present invention has a molecular weight of 400 kDa or more, 600 kDa or more, or 1,000 kDa or more.

In some embodiments, an irradiation-modified fibroin of the present invention exhibits radical scavenging performance that is improved by 3 times, 5 times, 7 times, or up to 10 times compared to a typical fibroin without irradiation, thereby enhancing physiological activities such as antioxidant activity. Radical scavenging performance can be measured, for example, by DPPH radical scavenging activity.

In some embodiments, an irradiation-modified fibroin of the present invention exhibits tyrosinase inhibitory effects improved by 1.5 times, 3 times, 5 times, or up to 7 times compared to a typical fibroin without irradiation, thereby enhancing whitening effects.

Melanin in a human skin is an important mechanism for protection of the skin from UV caused damage, however, can cause abnormal pigmentation such as melasma, freckles, senile lentigines (or actinic keratinosis) and over-pigmentation, leading to undesirable problems. Tyrosinase can act for biosynthesis of melanin in the skin and a tyrosinase inhibitor is well known as an important element used in manufacturing cosmetics to endow whitening effects thereto.

The irradiation-modified fibroin of the present invention can exhibit cytotoxicity to cancer cells increased to a maximum of 40 times that of a typical fibroin without irradiation, thereby enhancing cancer cell growth inhibitory effects.

In another aspect of the present invention, there is provided a method for production of a fibroin, comprising the step of: irradiating an initial fibroin with a radiation absorbed dose in the range of 1 kGy to 1,000 kGy, so as to produce the fibroin having a molecular structure modified by irradiation, wherein the produced fibroin has increased molecular weight and improved antioxidant activity, tyrosinase inhibitory ability and/or cytotoxicity to cancer cells.

The initial fibroin used herein can include a fibroin isolated from cocoons or a synthesized fibroin. The fibroin isolated from cocoons can be prepared by treating the cocoons in a sodium carbonate solution to remove sericin, heating the treated cocoons, dissolving the heated material in a three-component solution including CaCl₂-H₂O-ethanol, filtering the solution to remove impurities, and then, conducting dialysis of the filtered fibroin solution. The purified fibroin solution can undergo lyophilization to produce a fibroin product in the form of powder.

Alternatively, the synthesized fibroin can be produced by using micro-organisms to bio-synthesize the fibroin or through polypeptide synthesis.

The present inventive method for producing the fibroin can further comprise a step of lyophilizing the prepared fibroin by any conventional process.

Irradiation applied in the present invention can include at least one selected from a group consisting of γ-irradiation, electron beam irradiation and x-ray irradiation and, is preferably γ-irradiation or electron beam irradiation in view of an increase in molecular weight of the fibroin obtained by irradiation.

The modification of the molecular structure of the fibroin according to the present invention can be a decrease in an α-helix secondary structure, or otherwise, an increase in at least one secondary structure selected from a group consisting of β-sheet, β-turn and random coil.

The modification of molecular structure of the fibroin according to the present invention can be a decrease in an α-helix secondary structure, as well as an increase in at least one secondary structure selected from a group consisting of β-sheet, β-turn and random coil structures.

In some embodiments, the modification of molecular structure of the fibroin according to the present invention is a decrease in an α-helix secondary structure and, at the same time, an increase in each of β-sheet, β-turn and random coil type secondary structures, in view of maximum improvement in antioxidant activity, tyrosinase inhibitory ability and cytotoxicity to cancer cells.

In another aspect of the present invention, there is provided a method for using the fibroin with modified molecular structure as described above, as a raw material in any one of foods, cosmetics and medicines for enhancing antioxidant activity, tyrosinase inhibitory ability and/or cytotoxicity to cancer cells.

In a further aspect of the present invention, there is provided a composition including the fibroin with modified molecular structure as an active ingredient, which is useful for enhancing antioxidant activity, tyrosinase inhibitory ability and/or cytotoxicity to cancer cells.

Such methods for use of the fibroin as a raw material in any one of foods, cosmetics and medicines according to the present invention can be optionally varied within regulations of the Food Code, Food Additives Codex, and/or designation, standards and guidelines for testing cosmetic ingredients, and so forth.

Foods including the fibroin with modified molecular structure according to the present invention can include a food composition, for example, beverages, noodles, frozen foods, dairy products, meat products, seasoned foods, raw foods, etc., however, are not particularly limited thereto.

Cosmetics including the fibroin with modified molecular structure according to the present invention can include a cosmetic formulation, for example, lotion, cream, gel, etc., however, are not particularly limited thereto.

Medical products including the fibroin with modified molecular structure according to the present invention can include a medical formulation, for example, a tablet, a granule, a pill, an oral liquid, an injection, a cream, an ointment, etc., however, are not particularly limited thereto.

Such food compositions, cosmetic formulations and/or medical formulations can be manufactured by any conventional method without particular limitation thereof.

Hereinafter, exemplary embodiments of the present invention will be described in detail with reference to the following examples, which are only given for the purpose of illustration and not to be construed as limiting the scope of the invention.

### EXAMPLES

### Example 1

### Preparation of Fibroin with Modified Molecular Structure by γ-Irradiation 1

A natural fibroin isolated from cocoons was used in the present invention.

After treating 10 g of cocoons in 500 mL of 5% (w/v) concentration sodium carbonate solution, the treated solution was heated for 1 hour and filtered through a filter paper to remove sericin dissolved in the solution. Using hot water, the residue was washed several times so as to completely eliminate the residual sericin and sodium carbonate. As a result, a fibroin silk yarn containing fibroin alone without sericin moiety was obtained. This fibroin silk yarn was dissolved in a CaCl₂—H₂O-ethanol based three-component solution. The dissolved fibroin solution was placed in a dialysis membrane for dialysis over 3 days, followed by lyophilization to produce a fibroin product in the form of powder. This product was used in experiments.

A fibroin sample obtained as described above underwent irradiation using a cobalt-60 irradiator available from Advanced Radiation Technology Institute, Korea Atomic Energy Research Institute. A size of a radiation resource was about 300 kCi while an irradiation dose rate was 10 kGy/hour.

The radiation absorbed dose was detected using a 5 mm diameter alanine dosimeter available from Bruker Instruments (Rheinstetten, Germany) and a dosimetry system was first regulated in compliance with standards set by the International Atomic Energy Agency (IAEA) before use thereof.

After the sample, that is, silk fibroin was dissolved in distilled water to a concentration of 1 mg/mL, the solution was irradiated using a Co-60 γ-irradiation instrument IR-79 available from Nordion International Ltd. (Ontario, Canada) at an irradiation dose rate of 10 kGy/hour in order to reach the total radiation absorbed dose of 5 kGy, resulting in a fibroin solution with modified molecular structure.

### Example 2

### Preparation of Fibroin with Modified Molecular Structure by γ-Irradiation 2

A fibroin solution with modified molecular structure was prepared by the same procedure as described in Example 1, except that γ-irradiation was conducted to obtain the total radiation absorbed dose of 10 kGy.

### Example 3

### Preparation of Fibroin with Modified Molecular Structure by γ-Irradiation 3

A fibroin solution with modified molecular structure was prepared by the same procedure as described in Example 1, except that γ-irradiation was conducted to obtain the total radiation absorbed dose of 50 kGy.

### Example 4

### Preparation of Fibroin with Modified Molecular Structure by γ-Irradiation 4

A fibroin solution with modified molecular structure was prepared by the same procedure as described in Example 1, except that γ-irradiation was conducted to obtain the total radiation absorbed dose of 100 kGy.

### Example 5

### Preparation of Fibroin with Modified Molecular Structure by γ-Irradiation 5

A fibroin solution with modified molecular structure was prepared by the same procedure as described in Example 1, except that γ-irradiation was conducted to obtain the total radiation absorbed dose of 150 kGy.

### Example 6

### Preparation of Fibroin with Modified Molecular Structure by γ-Irradiation 6

A fibroin solution with modified molecular structure was prepared by the same procedure as described in Example 1, except that γ-irradiation was conducted to provide the total radiation absorbed dose of 200 kGy.

### Experimental Example 1

### UV Spectrum Analysis

Each of the silk fibroin solutions prepared by γ-irradiation according to Examples 1 to 6 was used for experiments and stored at 4° C.

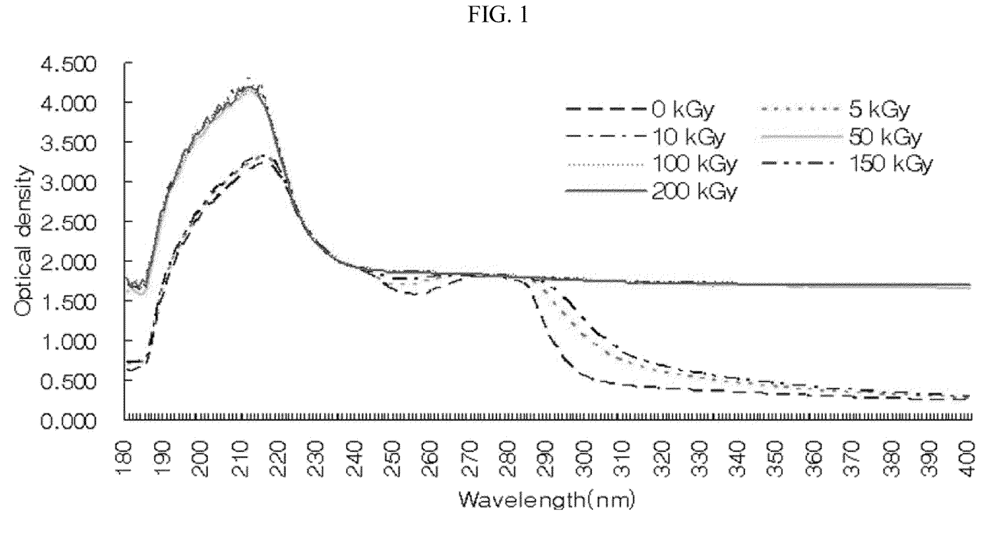

In order to determine structural modification of the silk fibroin by γ-irradiation, the silk fibroin solution was dissolved to have a concentration of 2 mg/mL, treated by γ-irradiation, and analyzed through UV-VIS spectrum at 180 nm to 400 nm using a UV spectrophotometer UV-1601 PC available from Shimadzu C. (Tokyo, Japan). The analysis result was shown in FIG. 1. As a control, a fibroin obtained without γ-irradiation was used.

UV absorption spectrum exhibited structural modification via absorbency of branched chains in an aromatic amino acid presented on a surface of the protein. Each of three amino acids such as phenylalanine, tyrosine and tryptophan has aromatic branched chains and the aromatic amino acid absorbs light at UV spectral regions, like most of compounds having bond rings.

Both tyrosine and tryptophan mostly have UV absorbencies at 280 nm. Especially, tryptophan has an absorption rate 100 fold higher than phenylalanine and phenylalanine is usually measured at 260 nm.

Referring to FIG. 1, an absorbency of the silk fibroin treated by γ-irradiation increases at 260 nm and 280 nm according to an increase in radiation absorbed dose. A variation in UV absorbency demonstrates that there is a structural modification by irradiation. A reason behind such structural modification is that amino acids in a protein such as tryptophan and tyrosine are exposed by structural breakup of the protein. Therefore, it can be seen that a turbidity of the protein is increased at 330 nm with increased irradiation dose.

According to reported studies, the maximum absorption spectral region was 214 nm and this result demonstrates that peptide bonds mostly absorb fibroin at a UV spectral region.

Consequently, the above results are supported by Experimental Example 1.

### Experimental Example 2

### Circular Dichroism Spectrum Analysis

Circular dichroism spectrum (hereinafter, referred to as “CD spectrum”) was measured using a Jasco J-715 spectro-polarimeter equipped with a 150 W xenon lamp, which is available from Japan Spectroscopic.

The UV spectrum was measured at 190 nm to 250 nm. A sample (0.2 mg/mL) in PBS solution at pH 7.2 was analyzed using a 1 mm cuvette after washing under nitrogen.

After repeating the analysis three times, an average was calculated from the analyzed values by subtracting a value measured for PBS, wherein a CD spectrum was represented by the residue ellipticity (units are degrees cm²/dmol).

CD spectroscopy was used to determine a absorption difference between left-handed polarized light and right-handed polarized light generated by structural unbalance, more particularly, can determine a secondary protein structure at a far-UV spectral region of 190 nm to 250 nm. A chromophore at this region is a protein bond and such protein bond can exhibit a specific CD spectrum with different sizes and shapes where the protein bond is in a regularly folded environment or is located in α-helix, β-sheet or random coil structures, respectively.

Two negative peaks formed at 208 nm and 220 nm, respectively can exhibit a protein with an α-helix secondary structure, while a protein with a β-sheet secondary structure is identified by a peak formed at 214 nm.

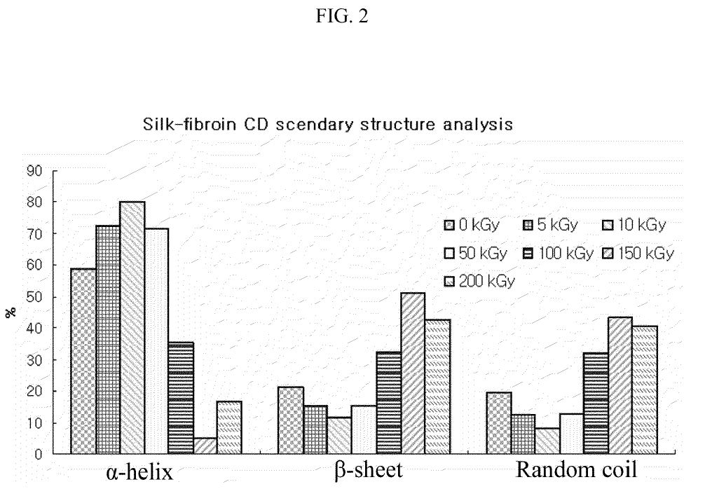

The fibroin has the secondary structure modified by irradiation and, as shown in FIG. 2, it can be seen that the α-helix secondary structure is reduced when an irradiation dose increases, while β-sheet and/or random-coil structures are relatively increased with a decrease in an α-helix secondary structure.

### Experimental Example 3

### Molecular Weight Analysis Using GPC

Gel permeation chromatography (GPC)-high performance liquid chromatography (HPLC) was used to determine a molecular weight of the fibroin treated by γ-irradiation.

A HPLC system used herein was a Waters Agilent HPLC system Mo. 2690 (MA, USA) equipped with a PL aquagel-OH column (with dimensions of 300×7.5 mm and 8 μm) available from Polymer Laboratories, Ltd. (UK).

A mobile phase of the above system was 0.1M sodium nitrate and the mobile phase passed through the column at a flow rate of 1 mL/min for 40 minutes. A pullulan standard for GPC was purchased from Showa Denko Co. to be used in this experiment.

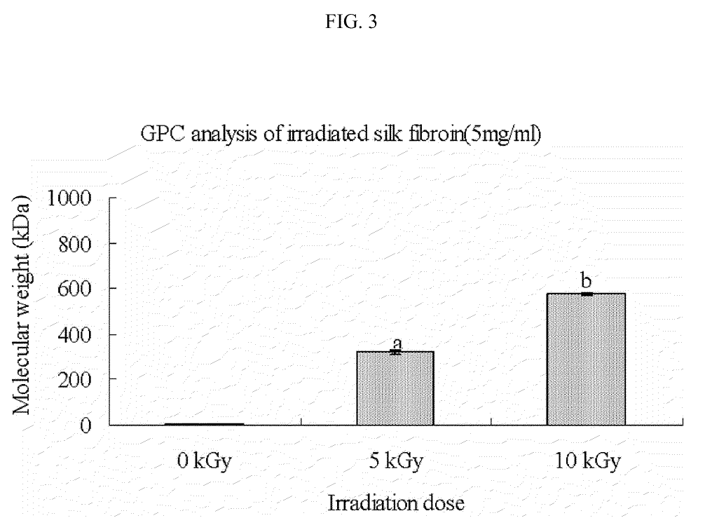

FIG. 3 illustrated a variation in molecular weight of a silk fibroin, which demonstrates irradiation effects at different does of irradiation. Referring to FIG. 3, a silk fibroin without irradiation has a molecular weight of not more than 5 kDa. However, silk fibroins irradiated at 5 kGy and at 10 kGy exhibited molecular weights of 320 kDa and 576 kDa, respectively. Such a result demonstrates that re-combination of molecules is increased by the structural modification where the irradiation dose increases.

### Experimental Example 4

### Determination of Radical Scavenging Performance of Fibroin with Molecular Structure Modified by γ-Irradiation

Electron donating performance of each fibroin sample prepared as in Examples 1 to 6 was determined according to BLOIS methods to detect hydrogen donation effects of a silk fibroin to 2,2-diphenyl-1-picryl-hydrazil (DPPH).

1 mL of 2×10⁴ M DPPH solution in 99% ethanol was added to 2 mL of each sample at a certain concentration and reacted at 37° C. for 30 minutes under vortex mixing. An absorbency of the reaction product was measured at 517 nm and electron donation effects were expressed as a difference in absorbencies determined as a percentage (%) before and after addition of the sample.

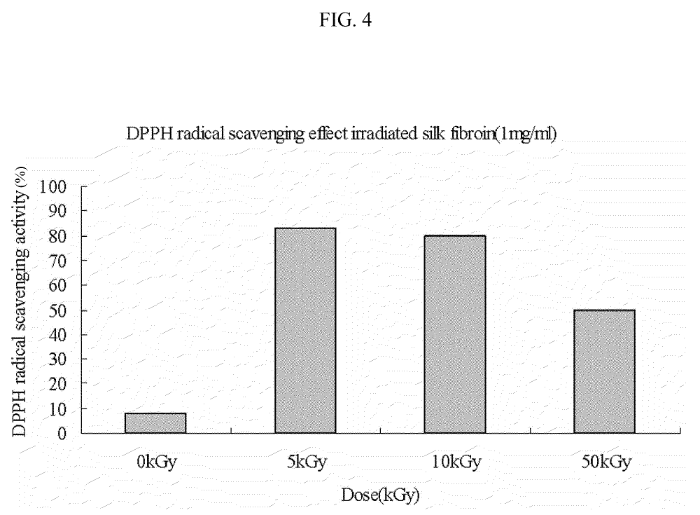

DPPH, a stable free group with absorptive properties at 517 nm, was used to examine radical scavenging performance of the silk fibroin. Antioxidant activity of the irradiated silk fibroin was shown in FIG. 4. Referring to FIG. 4, it was found that the irradiated silk fibroin has DPPH radical scavenging performance higher than that of the control at 0 kGy where both the fibroins have the same concentration and the antioxidant activity of the fibroin increases with increased irradiation dose.

### Experimental Example 5

### Determination of Tyrosinase Inhibitory Effects of Fibroin with Molecular Structure Modified by γ-Irradiation

Tyrosinase inhibitory effects of each fibroin sample prepared as in Examples 1 to 6 was determined in order to identify whitening activity of a silk fibroin treated by γ-irradiation.

After 10 mM L-3,4-dihydroxyphenylalanine (L-DOPA) (Sigma Chemical Co., St. Louis, Mo., USA) was dissolved in 0.5 mL of 0.175 M sodium phosphate butter (pH 6.8), 0.2 mL of the solution as a substrate was mixed with 0.1 mL of a sample solution to prepare a reactant solution. 0.2 mL of mushroom tyrosinase solution (100 U/mL, Sigma USA) was added to the reactant solution and reacted at 25° C. for 15 minutes. The obtained DOPA solution was subjected to measurement of absorbency of DOPA chrome in the solution at 475 nm.

Tyrosinase inhibitory activity was expressed as a decrease in absorbency as a percentage (%) when a sample was added, compared to a control without adding the sample.

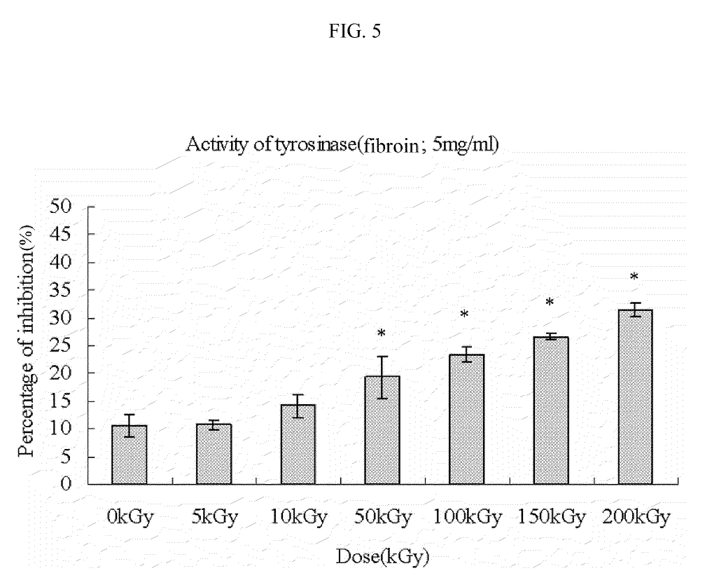

FIG. 5 illustrated tyrosinase inhibitory activity of the fibroin according to the present invention. Referring to FIG. 5, all of the γ-irradiated silk fibroins exhibited tyrosinase inhibitory activity higher than the silk fibroin without irradiation and it was found that the tyrosinase inhibitory activity is increased with increased irradiation dose.

### Experimental Example 6

### Determination of Cytotoxicity of Fibroin with Molecular Structure Modified by γ-Irradiation to Cancer Cells

Cytotoxicity of each fibroin sample prepared as in Examples 1 to 6 to cancer cells was determined in order to identify whitening activity of a silk fibroin treated by γ-irradiation.

B16BL6 (skin cancer), AGS (gastric cancer), HT-29 (colon cancer) and RAW 264.7 (macrophage cells) cell lines were obtained from Korean Cell Line Bank. The cell lines were cultured and used in experiments.

Both B16BL6 and RAW 264.7 cell lines were cultured in EMEM and DMEM media, respectively, with each of the media containing 100 U/mL penicillin, 100 U/mL streptomycin and 10% fetal bovine serum. On the other hand, each of AGS and HT-29 cell lines was cultured in RPMI 1640 medium containing 100 U/mL penicillin, 100 U/mL streptomycin and 10% fetal bovine serum at 37° C. using a 5% CO₂ incubator.

Cytotoxicity of a silk fibroin to cancer cells was evaluated by 3-(4,5-dimethylthiazolyl)-2,5-diphenyl-tetrazolium bromide (MTT) (Sigma) assay, compared to cytotoxicity to non-treated cells.

Each of the cancer cell and the normal cell was fed to each well of 96-well plate at a concentration of 3×104 cells/well. A silk fibroin sample at 5 mg/mL was added to the 96-well plate. After the plate was incubated at 37° C. for 24 hours, 30 μL of MTT reagent at 5 mg/mL was added to each of the wells, followed by culturing at 37° C. for 2 hours. Centrifuging the cultured solution at 2000 rpm for 3 minutes, a supernatant was removed from the treated solution. Adding 100 μL of dimethylsulfoxide (DMSO) to the resulting solution, the mixture was reacted at 37° C. for 5 minutes and was subjected to measurement of absorbency at 540 nm.

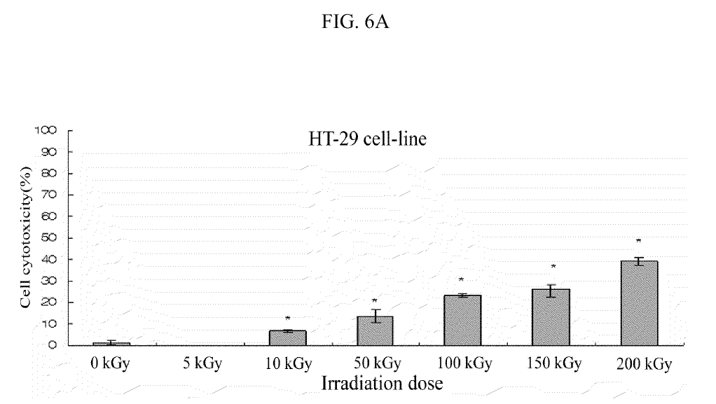

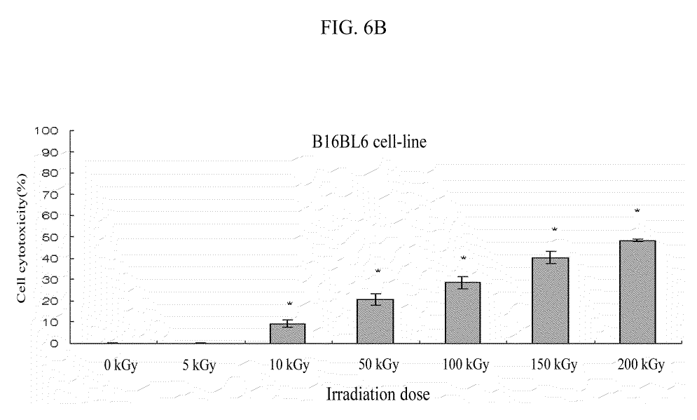

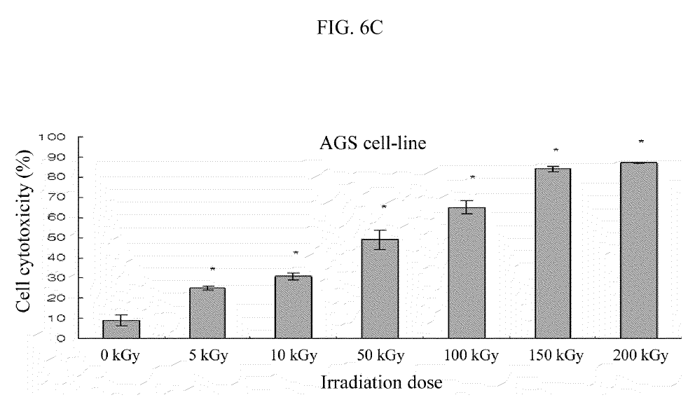

Cancer cell growth inhibitory effects of the irradiated silk fibroin were shown in FIGS. 6A-6C. In the present invention, HT-29 (colon cancer), B16BL6 (skin cancer) and AGS (gastric cancer) cell lines were used in the experiments.

In order to determine anti-cancer effects to the above cell lines, an MTT reduction assay was conducted to compare the present inventive fibroin samples with a control without irradiation, thus identifying the cancer cell growth inhibitory effects.

With increased irradiation dose, cytotoxicities to HT-29, B16BL6 and AGS cell lines were increased to 39%, 48% and 87%, respectively.

Comparing the cytotoxicity of the irradiated silk fibroin with that of the control without irradiation, it was found that the silk fibroins at all of irradiation doses exhibit higher cytotoxicity than the control. As a result, it is identified that irradiation can favorably improve resistance to cancer cells.

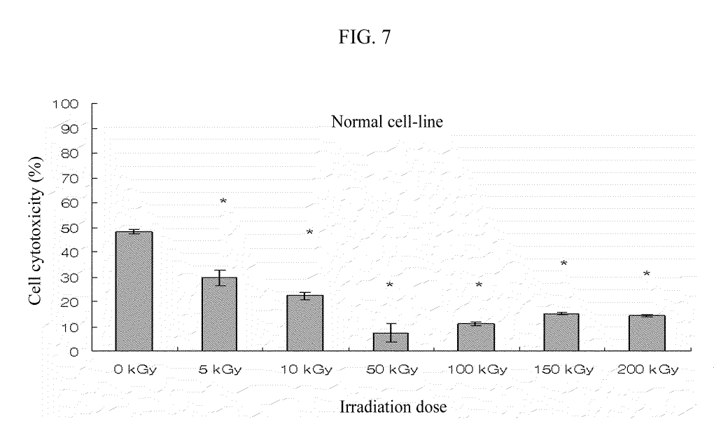

Cytotoxicity to cancer cells determined by the above experiments was enhanced with increased irradiation dose. In view of this fact, using RAW 264.7 cell line can determine cytotoxicity to normal cells. Chiarini et. al. have reported that a silk fibroin promotes human cell growth (A. Chiarini et al., “Silk fibroin/poly(carbonate)-urethane as a substrate for cell growth: in vitro interactions with human cells,” Biomaterial 24:789 (2003)). Based on the previous report, experimental results for effects of the irradiated silk fibroin to normal cells were shown in FIG. 7.

Referring to FIG. 7, illustrating measured results of a decrease in cytotoxicity of the irradiated silk fibroin to normal cells, cytotoxicities at 5 kGy, 10 kGy and 50 kGy were reduced to 29%, 22% and 7%, respectively, according to an increase in irradiation dose. However, when the irradiation dose reached 100 kGy or more, it was found that the cytotoxicity was not substantially increased or decreased.

From the above experiments, it can be seen that the irradiated silk fibroin shows very little cytotoxicity to normal cells, compared to the control. Comparing these results with the experimental results shown in FIG. 6, it can be seen that the cytotoxicity of the irradiated silk fibroin to cancer cells is improved while the cytotoxicity to normal cells is reduced.

Consequently, comparing the irradiated silk fibroin with the control in view of the above experimental results, it is understood that the irradiated silk fibroin exhibits improved antioxidant activity, whitening effects and cytotoxicity to cancer cells.

As is apparent from the above description, a high molecular weight fibroin with a specific molecular structure modified by γ-irradiation according to the present invention has superior physiological characteristics such as improved radical scavenging effects, tyrosinase inhibitory effects and/or cytotoxicity to cancer cells, as well as favorable whitening effects, compared to conventional materials without irradiation, thereby being effectively used as raw materials for foods, cosmetics, medicines, and the like.

### CONCLUSION

These examples illustrate possible embodiments of the present invention. While various embodiments of the present invention have been described above, it should be understood that they have been presented by way of example only, and not limitation. It will be apparent to persons skilled in the relevant art that various changes in form and detail can be made therein without departing from the spirit and scope of the invention. Thus, the breadth and scope of the present invention should not be limited by any of the above-described exemplary embodiments, but should be defined only in accordance with the following claims and their equivalents.

It is to be appreciated that the Detailed Description section, and not the Summary and Abstract sections, is intended to be used to interpret the claims. The Summary and Abstract sections can set forth one or more, but not all exemplary embodiments of the present invention as contemplated by the inventor(s), and thus, are not intended to limit the present invention and the appended claims in any way.

All documents cited herein, including journal articles or abstracts, published or corresponding U.S. or foreign patent applications, issued or foreign patents, or any other documents, are each entirely incorporated by reference herein, including all data, tables, figures, and text presented in the cited documents.

What is claimed is:

1. A modified fibroin having a molecular structure modified by irradiation, wherein the modified fibroin has an increased molecular weight compared to a naturally occurring fibroin and at least one property selected from: improved antioxidant activity, improved tyrosinase inhibitory ability, and improved cytotoxicity to cancer cells compared to a non-irradiated fibroin.

2. The modified fibroin of claim 1, wherein the irradiation is selected from: γ-rays, an electron beam, x-rays, and combinations thereof.

3. The modified fibroin of claim 1, wherein the modified fibroin has absorbed a radiation dose of 1 kGy to 1,000 kGy.

4. The modified fibroin of claim 1, wherein the modified fibroin has a decreased amount of α-helical secondary structure compared to a non-irradiated fibroin.

5. The modified fibroin of claim 1, wherein the modified fibroin has an increased amount a secondary structure selected from: β-sheet, β-turn, and random coil structures compared to a non-irradiated fibroin.

6. The modified fibroin of claim 1, wherein modified fibroin has a decreased amount of α-helical secondary structure and an increased in at least one secondary structure selected from a group consisting of β-sheet, β-turn and random coil structures compared to a non-irradiated fibroin.

7. The modified fibroin of claim 1, wherein the modified fibroin has a molecular weight of 10 kDa to 1,000 kDa.

8. A method for preparing a modified fibroin, the method comprising: irradiating an initial fibroin with a radiation absorbed dose of 1 kGy to 1,000 kGy to produce a modified fibroin having a molecular structure modified by irradiation, wherein the modified fibroin has an increased molecular weight and at least one property selected from: an improved antioxidant activity, an improved tyrosinase inhibitory ability, and an improved cytotoxicity to cancer cells compared to the initial fibroin.

9. The method of claim 8, wherein the initial fibroin is obtained by isolation from a cocoon or synthesis.

10. The method of claim 8, further comprising lyophilizing the modified fibroin.

11. The method of claim 8, wherein the irradiation is at least one selected from: γ-rays, an electron beam, and x-rays.

12. The method of claim 8, wherein the modification of the molecular structure is a decrease in an α-helix secondary structure.

13. The method of claim 8, wherein the modification of the molecular structure is an increase in at least one secondary structure selected from a group consisting of β-sheet, β-turn and random coil structures.

14. The method of claim 8, wherein the molecular structure of the modified fibroin exhibits a decrease in α-helix secondary structure, and an increase in at least one secondary structure selected from: β-sheet, β-turn, and random coil structures, compared to the initial fibroin.

15. The method of claim 8, wherein the molecular weight of the modified fibroin is 10 kDa to 1,000 kDa.

16. A product prepared by the process of claim 8.

17. The product of claim 16, wherein the product is selected from: a food product, a cosmetic product, and a medical product.

18. A composition comprising the modified fibroin of claim 1.

19. A composition comprising a modified fibroin, wherein the modified fibroin has a molecular weight of 10 kDa to 1,000 kDa and has a molecular structure that includes less than 50% of an α-helix structure, as determined by circular dichroism.

20. The composition of claim 19, wherein the modified fibroin has a molecular weight of 200 kDa to 1,000 kDa and has a molecular structure that includes 30% or more of a β-sheet structure and 30% or more of a random coil structure, as determined by circular dichroism.
